(19) United States
(12) Patent Application Publication
(10) Pub. No.: US 2022/0409068 A1
(43) Pub. Date: Dec. 29, 2022

(54) FATIGUE MANAGEMENT & OXYGENATION / HYDRATION OPTIMIZATION

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/839,095

(22) Filed: Jun. 13, 2022

Related U.S. Application Data
(60) Provisional application No. 63/209,813, filed on Jun. 11, 2021.
(60) Provisional application No. 63/209,810, filed on Jun. 11, 2021.

(51) Int. Cl. A61B 5/0205; G16H 40/67

(52) U.S. Cl. CPC A61B 5/02055; G16H 40/67; A61B 5/024

(57) ABSTRACT

Systems and methods for providing recommendations are provided for the proper timing and use of an electrolyte replacement device that is capable of delivering electrolytes to the bloodstream of a user through their buccal mucosa. At least one of; user characteristics, activity characteristics, activity context, or real-time physiological data about the user is used to determine fatigue level and the corresponding appropriate timing of delivery of doses of a composition with at least two nutrients and oxygen through the buccal mucosa.

### CROSS-REFERENCE TO RELATED APPLICATIONS

The present disclosure claims the priority benefit of U.S. provisional patent application No. 63/209,813 filed Jun. 11, 2021 and U.S. provisional patent application No. 63/209,810 filed Jun. 11, 2021, the disclosures of which are incorporated by reference herein

### BACKGROUND OF THE INVENTION

### 1. Field of the Disclosure

The present disclosure is generally related to fatigue management. More specifically, the present disclosure is related to fatigue management based on hydration, supplemental oxygen, and nutritional supplement delivery through the oral mucosa at a custom dosage and timing.

### 2. Description of the Related Art

Excessive fluid loss through sweat during intense exercise may leave an individual with an electrolyte deficiency in addition to dehydration. Low levels of electrolytes in the system can lead to cramping. Dehydration can negatively impact these measures as well as athletic performance. Further, an individual's oxygen and fluid consumption may vary widely depending upon the nature of their activity, the activity's context, and physical condition. A heavier individual exercising in high humidity may need significantly more water to remain at peak performance than they would in dryer conditions. Without the guidance of a professional trainer, it is difficult for an individual to know the optimal use schedule of these products for their personal activity level and the context of that activity before experiencing symptoms

Presently available products to counteract such effects include hydration products and supplemental oxygen. Salt pills, for example, provide large salt doses to attempt to overcome this issue with endurance athletes. This approach can oversupply the body with sodium however, to overcome the delay in bioavailability through the GI tract. A mechanism for delivering a smaller dose of electrolytes to the buccal mucosa may provide the necessary electrolytes with more rapid bioavailability.

During exercise, the body requires more oxygen than when the body is at rest. When oxygen cannot be delivered to the muscles faster to replace the used oxygen, the muscles will begin to convert the available glucose to lactic acid. Lactic acid will build up in the muscle, causing fatigue. More rapid delivery of oxygen to the system during exertion can reduce fatigue, increase mental acuity, and decrease reaction times.

Athletes will often try to “load” up on needed nutrients, such as carbohydrates or electrolytes, before certain activities. However, you can only ever load to 100%. Excess electrolytes will not be stored in the body, and instead, the salt lost through sweat must be replaced as the activity goes on.

Therefore, it would be desirable to monitor an individual's activity to assess fatigue and to make customized and context-specific recommendations that proactively allow a user to replace needed nutrients and/or adjust hydration and supplemental oxygen use so as to optimize athletic performance before exhaustion sets in.

### SUMMARY OF THE CLAIMED INVENTION

Systems and methods for providing recommendations are provided for the proper timing and use of an electrolyte replacement device that is capable of delivering electrolytes to the bloodstream of a user through their buccal mucosa. At least one of; user characteristics, activity characteristics, activity context, or real-time physiological data about the user is used to determine fatigue level and the corresponding appropriate timing of delivery of doses of a composition with at least two nutrients and oxygen through the buccal mucosa.

### BRIEF DESCRIPTIONS OF THE DRAWINGS

FIG. 1 illustrates an exemplary network environment in which a system for generating custom recommendations for fatigue management.

FIG. 2 is a flowchart illustrating an exemplary method for generating customized recommendations for fatigue management.

FIG. 3 is a flowchart illustrating an exemplary method for customizing recommendations.

FIGS. 4A-D illustrate exemplary correlations that may be detected by a system for generating custom recommendations for fatigue management.

FIG. 5 is a block diagram of an exemplary computing device that may be used to implement an embodiment of the present invention.

### DETAILED DESCRIPTION

Systems and methods for providing recommendations are provided for the proper timing and use of an electrolyte replacement device that is capable of delivering electrolytes to the bloodstream of a user through their buccal mucosa. At least one of; user characteristics, activity characteristics, activity context, or real-time physiological data about the user is used to determine fatigue level and the corresponding appropriate timing of delivery of doses of a composition with at least two nutrients and oxygen through the buccal mucosa.

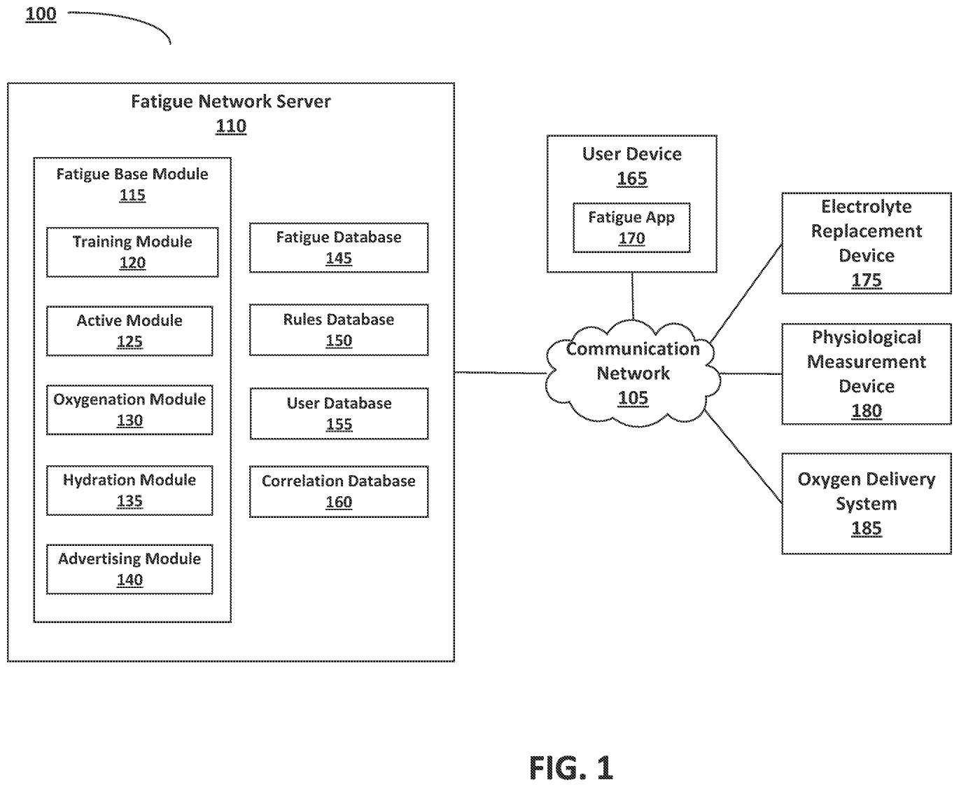

FIG. 1 illustrates an exemplary network environment 100 in which a system for generating custom recommendations for fatigue management. This network environment 100 may comprise a plurality of different computing devices communicating over a communication network 105. The computing devices may include a fatigue network server 110 that includes fatigue base module 115, training module 120, active module 125, oxygenation module 130, hydration module 135, advertising module 140, fatigue database 145, rules database 150, user database 155, and correlation database 160. Fatigue network server 100 may also be in communication with user device 165 (with fatigue management application 170), electrolyte replacement device 175, physiological measurement device 180, and oxygen delivery system 185.

Communication network 105 may be implemented using a collection of server devices to provide one or more services to coupled devices.

The fatigue network server 110 may communicate with a user's mobile device 165, and a physiological measurement device 180 through the communication network 105. In particular, fatigue network server 110 that may deliver instructions to a user for the proper timing for the use of oxygen delivery system 120 and hydrating during exertion to avoid, delay, and mitigate both muscle and cognitive fatigue related to dehydration and improve performance through proper oxygenation.

In one embodiment, the fatigue network server 110 contains a fatigue database 145, which may contain information about the system's users, such as exertion, fatigue, other physiological data, and electrolyte replacement. That data may be collected through user self-reporting, sensors in the mobile device 165 and in physiological measurement device(s) 180. That data may then be used by the fatigue base module 115 and active module 125 to inform the user on the proper timing of use for the electrolyte replacement device 175. In particular, fatigue network server 110 may deliver instructions the proper timing for the use of the electrolyte replacement device 175 during exertion to avoid, delay, and mitigate both muscle and cognitive fatigue related to an electrolyte deficiency related to sweating. It should be obvious to one skilled in the art that some or all of these functions could be combined and that some or all of these functions could be done on the user's mobile device 165.

Further, embodiments may include a fatigue base module 115, which may allow users to register with the fatigue network server 110 to get direction on the proper timing of use of the electrolyte replacement during periods of physical activity. In one embodiment, the fatigue network server 110 may provide a set of universal directions for all users. For example, users may be directed to utilize the electrolyte replacement device 175 at ten-minute intervals during the first 30 minutes of activity and at five-minute intervals after that. In another embodiment, the fatigue network server 110 may deliver more customized guidance to the user about electrolyte replacement device 175 use based on some amount of self-reported data. For example, directions may differ based on user weight, with larger individuals needing more frequent dosing or larger dosing than a smaller user. Directions may be further refined based on variables such as type of activity/level of exertion. For example, a user may be directed to use the electrolyte replacement device 175 at ten-minute intervals during moderate exercise and at five-minute intervals during strenuous exercise. The user may also report their perspiration level, with recommended use intervals for the utilize the electrolyte replacement device 175 varying based on the level of perspiration. A high perspiration level may result in a five-minute interval use recommendation and a low perspiration level resulting in a fifteen-minute use interval.

In one embodiment, the fatigue network server 110 contains a user database 155, containing information about the system's users, such as exertion, fatigue, other physiological data, and oxygen delivery system 185 usages. That data may be collected through user self-reporting, sensors in the mobile device 165, and physiological measurement device(s) 180. Data may also be collected from third-party providers, such as calendar applications, meteorological data providers, etc. That data may then be used by the fatigue base module 115 to inform the user on the proper timing of use for the oxygen delivery system 120 and hydration. Users with one or more physiological measurement devices 180 may receive recommendations based on direct physiological measurements, such as SPO2 levels, through the training module 120. Users without a physiological measurement device 180 may receive recommendations from the oxygenation module 130, hydration module 135, and advertising module 140 based on at least one context-based measure of the user's highly correlated activity hydration, oxygenation, or advertising need. It should be obvious to one skilled in the art that some or all of these functions could be combined and that some or all of these functions could be done on the user's mobile device 165.

Further embodiments may include a fatigue base module 115 that may allow users to receive recommendations about the proper timing of hydration, use of the oxygen delivery system 185, or ordering hydration or oxygenation supplies based on the current context. Users with a physiological measurement device 180 may have recommendations about hydration, oxygen delivery system 185 use, and supply ordering, based on direct measurement of the user's physiological condition. Users without a physiological measurement device 180 may still receive recommendations based on context characteristics highly correlated with physiological measurements that indicate hydration recommendations, oxygen delivery system 120 use, and supply ordering.

Training module 120 may allow users to receive recommendations about the proper timing of hydration, use of the oxygen delivery system 185, or ordering hydration or oxygenation supplies, based on the current context, including measurements from at least one physiological measurement device 180. Thresholds for physiological measures that may indicate the need for hydration, use of the oxygen delivery system 185, or ordering hydration or oxygenation supplies may be stored in the rules database 150. The context characteristics surrounding the physiological measure, such as the ambient temperature, altitude, user's location, age, weight, activity, etc., may be correlated with a data value for that measure that exceeds a recommendation threshold in the rules database 150. The correlations between context and physiological measures may be calculated by the training module 120 and stored in the correlation database 160.

Further embodiments may include an oxygenation module 130 that may allow users to receive recommendations on using the oxygen delivery system 185 based on one or more characteristics of the user's current context that correlates with a physiological measurement that would prompt a recommendation to take in oxygen. For example, data collected from training module 120 indicates that an SPO2 level below 88% is correlated with exercise time and altitude. A user with an elevated heart rate at an altitude above 5000 may receive a recommendation to take in oxygen. Further embodiments may include a hydration module 135 that may allow users to receive recommendations about fluid intake based on one or more characteristics of the user's current context that correlates with a physiological measurement that would prompt a recommendation to hydrate. For example, data collected from training module 120 indicates that a sodium concentration detected in a sweat sensor affixed to a subject's back greater than 80 mM is correlated with user weight and temperature. A user in a given weight range and exercising on a hot and humid day may receive a recommendation to take in water more frequently. Further, embodiments may include an advertising module 140 that may allow users to receive recommendations about the ordering of hydration and oxygenation supplies. Automated ordering systems and subscription services are well known in the art. The present invention may suggest ordering additional tanks of oxygen, tanks of a different size, or change ordering frequency based on context characteristics correlated with a change in consumption rate.

For example, a user may transition from working out two days a week to working three days a week. Accelerometers may detect this in the user's mobile device and prompt the advertising module 140 to suggest the user increase their standard order size by 50% to compensate. Further, embodiments may include a user database 155, which may contain all user data collected by the training module 120, the mobile device 165, physiological measurement device(s) 180, and contextual data from third parties weather data. This database may also contain historical analysis and recommendations previously made to individual users and users with at least one characteristic and the activity, and the context of that activity in common. Further, embodiments may include a correlation database 160, which may contain the correlations between physiological measurements collected by one or more physiological measurement devices 128 and one or more characteristic of the user, their activity, or their surroundings. In one embodiment, linear regression may calculate a correlation coefficient represented by an R-value between 0-1. A threshold R-value, such as 0.75, maybe pre-defined by a system administrator. Characteristics that correlate, with an R-value greater than 0.75, with a physiological measurement that exceeds a notification threshold in the rules database 150 may be used as a proxy for data from a physiological measurement device 180 to deliver recommendations to the user through the oxygenation module 130, hydration module 135, or the advertising module 140. Further, embodiments may include a rules database 150, which may the rules by which recommendations around hydration, oxygenation, and the ordering of supplies for both, may be provided to users based on some amount of data about the user, the activity the user is engaged in, the context of that activity, real-time physiological data about the user during the activity, or some combination of those factors. Further, embodiments may include an oxygen delivery system 185. Oxygen delivery systems, such as pressurized canisters with breath or pressure-actuated valves to provide oxygen to the user in concentrations greater than 22% in ambient air, are well known in the art.

In another embodiment, some or all of these factors may be combined into an algorithm. For example, users could identify in one of some number of weight classes, one of some number of perspiration levels, one of some number of exertion levels/times, and have a personalized dosing schedule based on combining two or more factors. This may result in a user over 200 lbs., who is engaged in a strenuous activity such as distance running, temperatures over 80 degrees, is recommended to utilize the electrolyte replacement device 175 at three-minute intervals. A user under 150 lbs., engaged in moderate activity such as jogging, in temperatures between 60-80 degrees, may be recommended to utilize the electrolyte replacement device 175 at twenty-minute intervals. In another embodiment, some or all of the data about the user and their activity may be collected by one of n number of physiological measurement devices 180 such as a microfluidic sensing patch for measuring sweat levels and electrolyte loss, a pulse oximeter, or another device that measures at least one physiological variable. It should be noted that mobile devices 165 may have the capability to collect some or all of this data. In such an embodiment, the user's heart rate may be used to measure the user's exertion level, a microfluidic sensing patch could measure electrolyte loss, and the user's mobile device 165 may be used to collect environmental data, such as temperature, humidity, etc. Environmental data may also be provided by a third party, such as a weather network. When a new user accesses the fatigue network server 110, the fatigue base module 115 may prompt the training module 120 to collect data from the user through self-reporting, sensor data, or a combination of the two. Once the fatigue network server 110 has collected sufficient data from the user to make recommendations on the utilization of the electrolyte replacement device 175, it will prompt the active module 125, which may actively monitor the user's condition and make recommendations on the utilization of the electrolyte replacement device 175 to minimize, mitigate or avoid fatigue. Further, embodiments may include a training module 120, which may collect data from the user through the fatigue app 170, the mobile device 165, the physiological measurement device(s) 180, or some combination thereof, to provide recommendations on the utilization of the electrolyte replacement device 175 that are customized to the user. In some embodiments, user data is reported through the fatigue management application 170 in the form of a questionnaire that may collect self-reported data about the user. This may be data about the user, such as, but not limited to, height, weight, age, gender, body mass index, etc. The self-reported data may also include data about the user's activity and the context of that activity, such as fluid, nutritional intake, physical activity, i.e., running a 5K, outside temperature, etc. This self-reported data may be used by itself to make recommendations. Sensor data from the mobile device 165 and the physiological measurement device(s) 180 may be used instead of or combined with user self-reported data. The training module 120 may identify what data is available from a given user, and collect that data from the user, store that data in the fatigue database 110. When some amount of data is collected from the user, the active module 125 may be prompted to make recommendations to the user about using the electrolyte replacement device 175. Further, embodiments may include an active module 125, which may utilize data in the fatigue database 110 to make recommendations to a user about the timing and use of the electrolyte replacement device 175. In one embodiment, the data collected is once, or at specific intervals, at the user is given a dosing schedule based on the data collected. For example, a user reporting their height, weight, and age along with the intended activity and its duration, such as a 35-year-old man who is 6′3″ and weighs 240 lbs., who will run a 5K for 30 minutes. A table in the fatigue database 110 may indicate that a user should utilize two doses of the electrolyte replacement device 175 at 4-minute intervals during the run, based on the provided data. The same table may recommend a single dose every 8 minutes for a 5′4″ woman who weighs 115 lbs. and is engaged in the same activity under the same conditions. In an exemplary embodiment, the active module 125 will collect data in real-time from the mobile device 165, the physiological measurement device(s), or some combination, to provide the user with recommendation on the use of the electrolyte replacement device 175 to prevent, mitigate, and minimize muscle and cognitive fatigue brought on by an electrolyte deficiency. Recommendations may be delivered to the user through the fatigue management application 170. Further, embodiments may include a fatigue database 110, which may contain all user data collected by the training module 120, the mobile device 165, and physiological measurement device(s) 180 and contextual data from third parties, such as weather data. This database may also contain historical analysis and recommendations previously made to individual users and users with at least one characteristic and the activity, and the context of that activity in common. Further, embodiments may include a rules database 112, which may contain the rules by will recommendations are provided to users based on some amount of data about the user, the activity the user is engaged in, the context of that activity, real-time physiological data about the user during the activity, or some combination of those factors. Further, embodiments may include an electrolyte replacement device 175 that may be a breathing apparatus or spray device that delivers oxygen in combination with at least two nutrients to the user by absorbing through the buccal and sublingual mucosa. Transmucosal Nutritional Supplements (electrolyte/other supplements) may benefit from replacing nutrients lost through excessive fluid loss from physical activities. Transmucosal delivery of nutrient supplements offers advantages over oral delivery when negative issues relating to the gastrointestinal tract, stomach, substance digestion, absorption, swallowing, protocol compliance, substance effectiveness, and other gastrointestinal metabolism issues are considered. The formulation or composition would include a nutritional supplement matrix fraction, (ii) a gas fraction, (iii) an enhancer fraction, (iv) a liquid fraction, and (v) a preservative fraction wherein the nutritional supplement matrix fraction, the gas fraction, the enhancer fraction, the liquid fraction, and the preservation fraction are all mixed or combined and treated to maintain a state of balanced suspension among the oxygen molecules for a specific duration of time before being dispensed from a canister. Furthermore, the composition may be in a compressed state in a canister before being dispensed. In another embodiment, the formulation or composition would include a nutritional supplement matrix fraction, (ii) a gas fraction, (iii) an enhancer fraction, (iv) a liquid fraction, and (v) a preservative fraction wherein the nutritional supplement matrix fraction, the enhancer fraction, the liquid fraction, and the preservation fraction are all mixed or combined and stored separately from the gas fraction in a canister before being dispensed. Additionally, the nutritional supplements are formulated, treated, and mixed with the gas fraction. The gas fraction is oxygen to maintain a state of balanced suspension among the oxygen molecules for a specific time after being dispensed. Furthermore, the composition may be in a compressed state in a canister before being dispensed. The transmucosal delivery of the supplement matrix is more efficient when the composition is atomized. It allows the enhancer fraction of the composition to adhere more to the nutritional supplement to the mouth's mucosal membranes. The buccal mucosa offers a promising administration site for nutrients as it has a rich blood supply and is relatively permeable. The bioadhesion of the delivery system is crucial for delivering across the buccal mucosa. Saliva may wash the delivery method off of the buccal region. To alleviate this, hydrophobic patches have been used to hold the nutrient or medication administered through the buccal mucosa. Buccal spray devices have been used to deliver insulin in a mist of fine droplets onto the mucin layer of the mucosal membrane. Without a hydrophobic layer to protect the desired deliverable from the saliva, it is crucial to atomize the mixture and deliver it at a high velocity directly onto the buccal mucosa to maximize the deliverable amount absorbed. Further, embodiments may include a communication network 105 implemented using a collection of server devices to provide one or more services to coupled devices. Further, the communication network 105 may be coupled to the fatigue network server 110, and one or more mobile device(s) 165 and physiological measurement device(s) 180. Further, the communication network 105 may be a wired and a wireless network. The communication network 105, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques, known in the art. The communication network may allow ubiquitous access to shared pools of configurable resources and higher-level services that can be rapidly provisioned with minimal management effort, often over the internet, and rely on sharing resources to achieve coherence and economies of scale, like a public utility. At the same time, third-party clouds enable organizations to focus on their core businesses instead of expending resources on computer infrastructure and maintenance. Further, embodiments may include a mobile device 165 such as a computing device, laptop, smartphone, tablet, computer, smart speaker, or I/O devices. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers. Devices may include a combination of multiple input or output devices, including, e.g., Microsoft KINECT, Nintendo Wii mote for the WIT, Nintendo WII U GAMEPAD, or Apple iPhone. Some devices allow gesture recognition inputs by combining some of the inputs and outputs. Some devices allow for facial recognition, which may be utilized as an input for different purposes, including authentication and other commands. Some devices allow for voice recognition and inputs, including, e.g., Microsoft KINECT, SIRI for iPhone by Apple, Google Now or Google Voice Search. Additional mobile devices have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality, including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or a wall, and may also interact with other electronic devices. Some I/O devices, display devices, or groups of devices may be augmented reality devices. An I/O controller may control the I/O devices. The I/O controller may control one or more I/O devices, such as, e.g., a keyboard and a pointing device, e.g., a mouse or optical pen. Furthermore, an I/O device may also allow storage and an installation medium for the computing device. In still other embodiments, the computing device may allow USB connections (not shown) to receive handheld USB storage devices. In further embodiments, an I/O device may be a bridge between the system bus and an external communication bus, e.g., a USB bus, a SCSI bus, a FireWire bus, and an Ethernet, a Gigabit Ethernet bus, a Fiber Channel bus, or a Thunderbolt bus. Further, the mobile device 165 could be an optional component. It would be utilized in a situation in which the paired wearable device utilizes the mobile device 165 as additional memory or computing power or connection to the internet. The mobile device 165 may serve as the connection to the communication network 105 for the physiological measurement device(s) 180. Further, embodiments may include a fatigue management application 170, which allows the user to provide information, or access to sensors in the mobile device 165 and the physiological measurement device(s) 180 to the fatigue base module 104.

The fatigue management application 170 may also deliver notifications or instructions to the user for dosage and timing of the electrolyte replacement device 175, as well as the proper hydration timing, use of the oxygen delivery system 120, or ordering hydration or oxygenation supplies. The notifications may be text, audio, visual, and haptic. Further, embodiments may include some number 1-n of physiological measurement devices 180 that may provide data about the user, the user's activity, and the context of the user's activity, that may be used to measure, analyze, and predict fatigue, performance, and recovery. In one embodiment, the sensors are located in the mobile device 165. They may include pedometers or location tracking to measure distance traveled over time, and the mode of movement, such as cycling or running. In another embodiment, some microfluidic sensing patches 1-n may be worn at various parts of the body for the regional measurement of the composition and dynamics of exercise and iontophoretic sweat. This may allow for sweat, in the form of NA+ or K+, and secretion rates to be measured and used to predict whole-body fluid loss and predict fatigue.

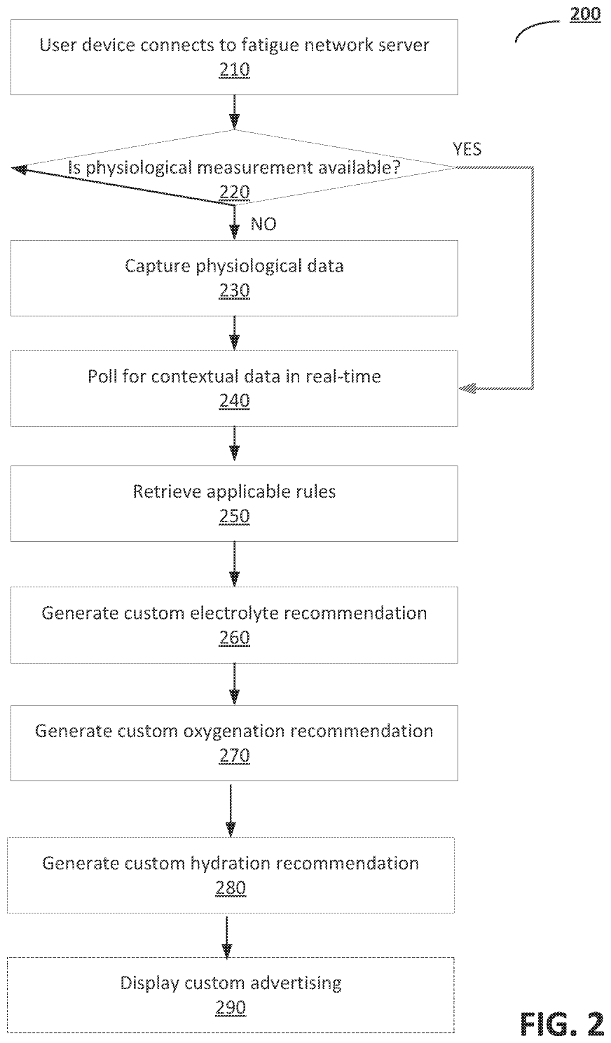

FIG. 2 is a flowchart illustrating an exemplary method 200 for generating customized recommendations for fatigue management. Method 200 may be performed based on execution of the instructions stored in the fatigue base module 115. The process may begin with a user device 165 (with fatigue management application 170) connecting, at step 210, to the fatigue network server 110. This module may determine, at step 220, if data is needed from the user to allow the system to generate recommendations for the timing and dosage of the electrolyte replacement device 175. In some embodiments, this is data from physiological measurement device 180 regarding new users that may include characteristics of the user such as height, weight, age, gender, fitness level, sweat level, etc. In such an embodiment, the user may provide this data once when initially registering with the fatigue network server 110 and connecting physiological measurement device(s) 180. Suppose the user has already registered with the fatigue network server 110 and utilizes at least one of a mobile device 165 and/or physiological measurement device(s) 180 to get real-time recommendations from the active module 125. For example, a microfluidic sweat sensor may be attached to the user's back and have a short-range connection, such as Bluetooth, to the mobile device 165

In step 220, it may be determined that there may have already been collected a statistically significant sample size of physiological data. If not, this method 200 will proceed to step 230, triggering one or more physiological measurement device(s) 180 to capture physiological data. If no more data needed, the method 200 will proceed to step 240, in which the training module 120 may be executed to poll for contextual data in real-time. In one embodiment, the user's mobile device 165 may be polled for related to the user's activity and its context. For example, an accelerometer in a smartphone may provide step counts. The GPS may provide geolocation, and the microphone or camera may be used to determine the activity the user is engaged in. In one embodiment, third-party data, such as from a weather service, may be used for data related to temperature, humidity, precipitation, etc. One embodiment data related to user activity may come from the user's mobile device 165 being connected with smart equipment, such as a stationary bike or treadmill.

In some embodiments, the user will provide data each time they engage in physical activity to produce context-specific recommendations. For example, in addition to information about the user, the user may provide information about the activity, such as a 5K run, and the context of the activity, 92 degrees and sunny. In another embodiment, real-time data may be collected about the user from the mobile device 165 and physiological measurement device(s) 180. In such an embodiment, the training module 120 may collect data about the user's activity for some time until there is a statistically significant sample size used by the active module 125 to make context and user-specific recommendations about the timing and dosage of the electrolyte replacement device 175.

Once the training module 120 collects the contextual data necessary for a given embodiment in step 240, this method 200 may proceed to step 250 in which the active module 125 may retrieve applicable rules. The active module 125 may use the applicable rules to generate one or more recommendations in steps 260-290 about the timing and dosage of electrolytes via electrolyte replacement device 175, supplemental oxygen via oxygen delivery system 185, and/or hydration, as well as delivery custom advertising.

In steps 260-290, fatigue network server 110 may execute oxygenation module 130, hydration (including hydration associated with electrolytes) module 135, and advertising module 140. The oxygenation module 130 may generate a custom recommendation for the user about customized settings and/or usage of the oxygen delivery system 185 based on the data received. The hydration module 135 may similarly generate a custom recommendation for the user regarding amount and timing of hydration and electrolytes based on the data received. The advertising module 140 may recommend the user about the ordering of oxygenation and hydration (including or not including type or amount of electrolytes) supplies based on the data received.

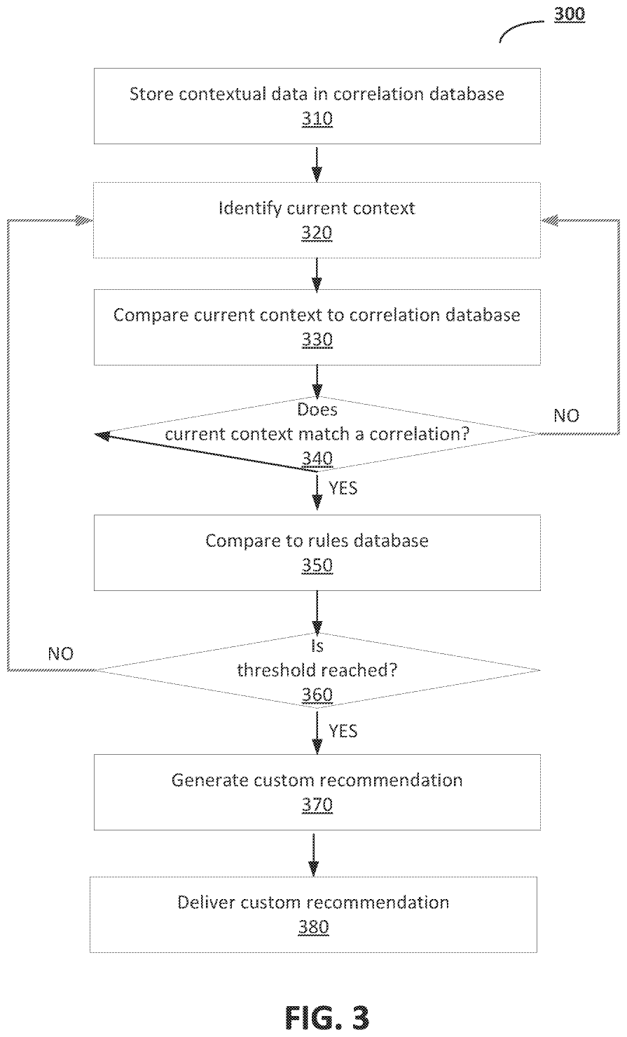

FIG. 3 is a flowchart illustrating an exemplary method 300 for customizing recommendations. Method 300 may be performed based on execution of the training module 120. At step 310, contextual data may be collected and stored in correlation database 310. For example, training module 120 may determine if the user is a new or existing user to the fatigue network server 110. If the user is unique to the fatigue network server 110, this module will collect the new user data via one or more sensors associated with the user device(s) 165 and/or physiological measurement device(s) 180. This information may include, but is not limited to, the user's contact information, age, weight, height, gender, fitness level, sweat level, diet, sleep patterns, mobile device(s) 165, physiological measurement device(s) 180. The collected data may be written to the fatigue database 145. In another example, the user may enter their age, height, and weight (which may be used to determine and customize one or more dosage and timing recommendations).

Step 320 may occur upon a prompt from the fatigue base module 110 requesting a recommendation on the dosage and timing of the use of the electrolyte replacement device 175 (or oxygen delivery system 185). In step 320, a current context may be identified. For example, the current context may indicate that a user utilizing at least physiological measurement device 180 has logged into the fatigue management application 180. A connection may be established to the physiological measurement device(s) 180. The physiological measurement device 180 may be polled for new contextual data. It may also be determined if new data is available from the physiological measurement device 180 or other real-time data available from the user, the mobile device(s) 165, and physiological measurement device(s) 180. If there is real-time data available, the device(s) and data available is identified. For example, the user data may include the location and step data available from his mobile device 165 and two physiological measurement devices 180, a smartwatch that provides heart rate data, and a microfluidic sensing patch that includes fluid and electrolyte loss data. These devices and data points may be recorded in the fatigue database 145 for use by the active module 125 in providing real-time recommendations to the user.

In step 330, the current contextual data received may then be compared to correlation database, and in step 340, it may be determined as to whether the current context matches a correction. If not, the method 300 may revert back to step 320 to further analyze and collect data regarding current contexts. If there is a match, the method 300 may proceed to step 350 where the matching context may be compared to the rules database 150 (e.g., the notification thresholds for that data type). For example, the physiological data value correlated with at least one current context characteristic may be compared to the rules database 150. It may be determined at step 350 if the current context is correlated with a data value that exceeds the threshold identified in the rules database 150. If the current context is not correlated with a physiological measure that meets or exceeds a threshold in the rules database 150, it proceeds to step 370. If the current context is correlated with a physiological measure that exceeds a threshold in the rules database 150, for example, a custom notification to hydrate may be generated in step 370 and delivered in step 380.

In another implementation of steps 340 and 350, the average weekly temperature (85 degrees Fahrenheit) for the user's current geolocation may be correlated (an R-value of 0.81) with an increase of 5% in the user's hydration needs. The threshold for identifying a current context characteristic as correlated may be defined by a system administrator. For example, only correlations with an R-value greater than 0.75 may be considered correlated. In one embodiment, the threshold for correlation may be calculated by an adaptive algorithm. The change in consumption correlated with at least one current context characteristic may be compared at step 350 to the rules database 150. It may be determined if the current context is correlated with a data value that exceeds the threshold identified in the rules database 150. If the current context is not correlated with a change in consumption that exceeds a threshold in the rules database 150, the method 300 may return to step 320. If the current context is correlated with a change in consumption that exceeds a threshold in the rules database 150, a notification to order supplies or change the quantity or frequency of standing orders may be generated in step 370 and delivered in step 380.

In step 360, it may be determined whether a threshold associated with the matching rule may be met. Depending on the type of contextual data and matching rules thereto, the thresholds may be set at different levels. For example, an SPO2 level of 87% may be received from a user with a pulse oximeter is one of their physiological measurement devices 128. This may be compared to the notification threshold of an SPO2 level of 88%. It may be determined at step 310 if the received data value exceeds the threshold associated with that data value in the rules database 118. For example, the received SPO2 level of 87% exceeds the threshold in the rules database 118, indicating any values below the threshold should receive a notification from taking in oxygen from the oxygen delivery system 120 if the threshold has not been reached.

In another example, the temperature (92 degrees Fahrenheit) and humidity level (25%) in which the user has been may be correlated (an R-value of 0.89) with the user experiencing a whole-body fluid loss rate of greater than 6 g/min. This measure may be collected and calculated with one or more microfluidic sweat sensors, as described in “Regional and correlative sweat analysis using high-throughput microfluidic sensing patches toward decoding sweat” by Nyein et al. 1. The threshold for identifying a current context characteristic as correlated may be defined by a system administrator. For example, only correlations with an R-value greater than 0.75 may be considered correlated. In one embodiment, the threshold for correlation may be calculated by an adaptive algorithm. If no current context characteristics meet the correlation threshold, the method 300 may return to step 320.

Where the threshold is met, the method 300 may proceed to step 370. If the received data value exceeds the identified threshold in the rules database 150, a custom recommendation may be generated in step 370 and delivered to the user device 165 at step 380. For example, the contextual data may be characteristics of the user, such as age, sex, weight, etc., characteristics of the activity the user is engaged in, such as the activity type, duration, location, etc., or characteristics of the environment of the activity, such as altitude, temperature, humidity, etc. The matching correlations in correlation database 160 may include the relationship between various context characteristics and the physiological measurements received. In one embodiment, this relationship may be calculated through linear regression and expressed as an R-value between 0 and 1. There are many ways known in the art for correlating data, such as convolution, cross-correlation, etc.

All records in the correlation database 160 with at least one similar characteristic to the current context are identified. For example, the current SPO2 measure was collected at an elevation of 5000 feet after 5 minutes of a heart rate above 120 bpm. The best fit line may be plotted through all the historical data collected that compared altitude to SPO2 measurements after at least 5 minutes of an elevated heart rate. The correlation database 160 may be updated with the new R-value.

In step 370, these custom recommendations may be generated, and in step 380, the custom recommendations may be delivered to a designated user device 165 through the fatigue management application 170 after receiving the user's response to these questions. In other embodiments, the recommendations may be in the form of written instructions, such as a table with height and weight that functions similarly to a body mass index table. For example, a 6′1″ user who weighs 200 lbs. may be recommended to use the electrolyte replacement device 175 every 10 minutes during activity. In other embodiments, the user will receive recommendations based on data about a given activity, such as moderate or strenuous activity levels, high and low heat, sun, humidity, etc. In these embodiments, the same user may see a recommendation to use the electrolyte replacement device 175 every 5 minutes in high heat conditions. It should be obvious to one skilled in the art that these recommendations could be based on any number of combinations of these characteristics of activity. For example, a heavier user who is in a high heat environment and is engaged in a strenuous activity may see a recommendation to use the electrolyte replacement device 175 every 2 minutes.

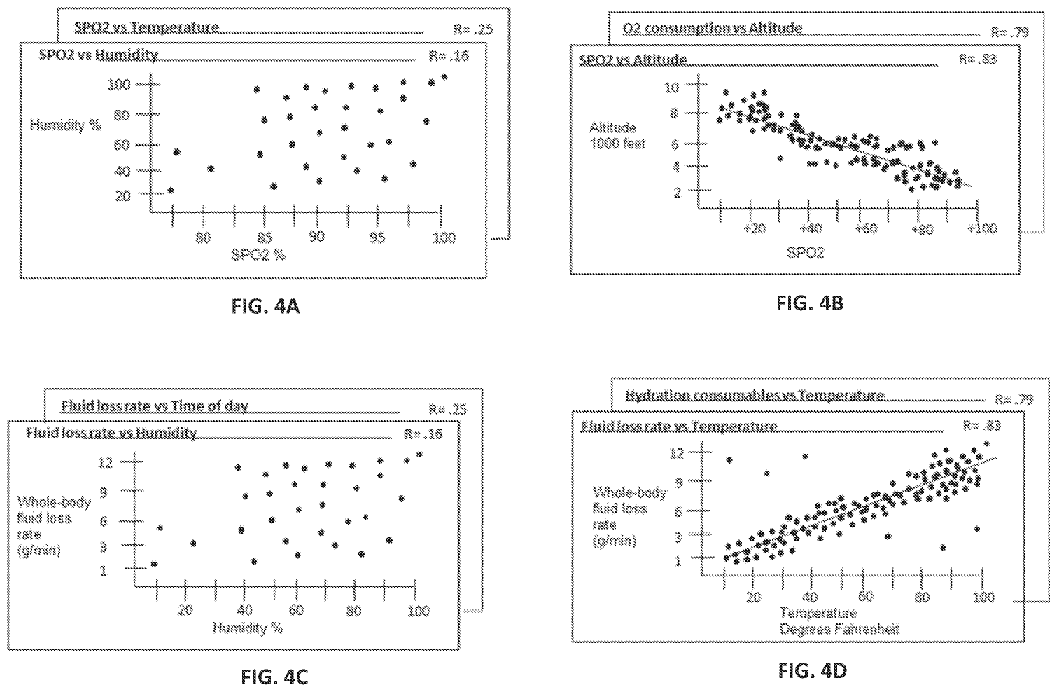

FIG. 4 illustrates the “Active Module.” The process begins with this module receiving, at step 400, a prompt from the fatigue base module indicating a user is connected to the fatigue network server 110, and some amount of data related to the user is available to be used in producing a recommendation about the timing and dosage for the use of the electrolyte replacement device 175. This module will identify, at step 402, the data available from which a recommendation can be made. In some embodiments, this data will be in the fatigue database 110 and may include a user's age, weight, height, gender, etc., sensor data from mobile device(s) 165, physiological measurement device(s) 180, and may also include data about the activity, and the context of the activity. Table 1 below illustrates an exemplary set of fields in a fatigue database:

TABLE 1

| | | | | Dwayne |
|---|---|---|---|---|
| USER | Hector Camacho | Joe Bauers | Rita Randolph | Elizondo |
| AGE | 40 | 35 | 28 | 45 |
| WEIGHT | 210 lbs | 180 lbs | 125 lbs | 260 lbs |
| HEIGHT | 5′11″ | 6′11″ | 5′4″ | 6′5″ |
| GENDER | M | M | F | M |
| FITNESS | High | High | Medium | Low |
| LEVEL | | | | |
| SWEAT LEVEL | High | High | Medium | Low |
| MOBILE | MobileID123 | MobileID234 | MobileID345 | MobileID456 |
| DEVICE | | | | |
| STEPS | Not Applicable | MobileID234/ | MobileID345/ - | Not Applicable |
| DEVICE/DATA | | JBsteps.dat | RRsteps.dat | |
| HEART RATE | WatchID567/ | WatchID789/ | WatchID678/ | Not Applicable |
| DEVICE/DATA | HCheartrate.dat | JBheartrate.dat | RRheartrate. dat | |
| SWEAT RATE | Not Applicable | MFDID123/ | Not Applicable | Not Applicable |
| DEVICE/DATA | | Jbsweat.dat | | |

The database illustrated in Table 1 above may contain data about each user of the fatigue network server 110, as collected by the training module 120. This may include age, height, weight, gender, etc. This may also include self-reported data about the user's health and fitness, such as overall fitness level, how sweaty they are when they exercise, how frequently they exercise, their diet, sleep patterns, etc. While in this example, the user-defined their fitness level on a high medium or low scale. It should be obvious to one skilled in the art that a fitness level could be calculated based on the user's response to a series of questions/or sensor measurements. The database may also contain data related to each mobile device associated with each user. The database may also contain real-time sensor data available, what mobile device 165 or physiological measurement device 180 that data is available from, and a data file of the historical measurements of that data for the user. For example, user Camacho has a smartwatch that provides heart rate data. The user Camacho's heart rate data over time is stored in the corresponding data file. That data file may also include the context of the measurement, including but not limited to the time and location of the measurement, the current activity, and any other sensor data collected at the same time.

For example, a user Bauers may be a 35-year-old male who is 6′1″ tall and weighs 175 lbs. The user Bauers is about to run a 5K on a 95-degree day and has positioning and step counting data available from a smartphone, heart rate data from a smartwatch, and sweat data from a microfluidic patch. This module then retrieves, at step 404, a recommendation from the rules database 112 based on some or all of the available data. In one embodiment, the user may look at a printed table that may come with the electrolyte replacement device 175 that shows height on one axis and weight on the other, with recommended use intervals in each of the crosses. These baseline recommendations could range from every 12 minutes for a short and light user and every 8 minutes for a taller, heavier user. In another embodiment, the user may have provided many responses through the training module 120 that may be used to provide a recommendation that is more personalized to the user. For example, the user Bauers may indicate a low fitness level (on a low/med/high scale), and the baseline recommendation frequency/dosage may be increased. Other factors about the user, such as sweat level (are they a heavy sweater), diet, sleep habits, etc., could be used to increase or decrease the baseline recommendation given to the user. Both the baseline recommendation and this more personalized version are meant to apply broadly to all types of physical activities, regardless of context. In another embodiment, either the baseline recommendation or the personalized baseline recommendation could be further adjusted based on the activity's activity and context. For example, the recommended use interval may be shortened by 10% for every 5 degrees above 80 degrees due to increased fluid loss as temperature increases. A dosage change could be used in place of or in conjunction with an interval change. Similar adjustments could be made based on other aspects of the context of the activity. For example, low humidity or high altitude contribute to an increase in fluid loss rate from sweating. Sensors in the mobile device 165, or a third-party provider, may provide temperature, altitude, humidity, location, and other context factors for the activity. Aspects of the activity may also be considered. For example, a person loses less fluid swimming as they do running, so the use interval for a swimmer may be longer. The exertion level for an activity may also be considered, with more strenuous activities calling for more frequent use or greater doses. The duration of the activity may also be considered. For example, a marathon runner may have a more frequent use interval than someone running a 5K due to the expected salt loss from the longer duration activity. This module may determine, at step 406, if there is real-time data available about the user/user's activity. For example, the user Bauers has sweat data from a microfluidic sensing patch, heart rate data from a smartwatch, and step/positioning data from their smartphone. At step 408, this module may pollute the available real-time data sources for new data events. This module may then determine, at step 410, if the real-time data is outside of the acceptable range. It should be obvious to one skilled in the art that there are many different ways to determine an acceptable range for a given parameter. For example, the user Bauers' heart rate, as measured by his smartwatch, may be compared to the target heart rate zone, defined by the American Heart Association, for his age. The personalized or baseline recommendations may be kept the same, while Bauer's heart rate remains between 50-85% of the maximum recommended for the age group. For a 35-year-old, the target heart rate zone is between 93-157 beats per minute (bpm), with 50-85% of the maximum being the ideal range. In another embodiment, heart rate data for user Bauers may be measured over a while. Their average heart rate during activity could be calculated. The baseline or personalized recommendation may be considered inside the acceptable range when it is within one standard deviation of their average heart rate during activity. A similar calculation may be applied to salt levels in the user's sweat as measured by the microfluidic sensing patch. Within one standard deviation of the average amount of NA+ or K+ detected, it is the acceptable range. One standard deviation is just one example of an acceptable range. For different variables, users, or applications, the acceptable range may be larger or smaller. This module may then provide a recommendation update at step 412 if the real-time data is outside of the acceptable range. The notification may be a haptic reminder at the appropriate interval for using the electrolyte replacement device 175. The notification may be a text or audio message that the recommended use interval or dose has changed in other embodiments. For example, the user Bauers has a baseline recommended use interval of 10 minutes based on his height and weight. It is personalized to every 8 minutes based on his self-reported fitness level of low. The user Bauers may have an average heart rate of 125 bpm, with a standard deviation of 15, based on historical data collected through a mobile device 165 and physiological measurement device 122. In one embodiment, the user Bauers may receive a notification when his heart rate exceeds 140 bpm (average of 125 plus one standard deviations 15) to increase his use of the electrolyte replacement device 175 every 7 minutes. In another embodiment, the user Bauers may receive a haptic notification at the recommended use interval. That interval would be adjusted, at step 412, to the new level based on the real-time data. This module may then determine, at step 414, if the activity is complete. This may be determined through the user's activity level as indicated by either the mobile device(s) 165 and the physiological measurement device(s) 180. The user is no longer running after completing a 5K. In another embodiment, the user may indicate the activity is over through the fatigue management application 170. If the activity is not complete, this module may return to step 408 and poll for additional real-time data. This looping function may enable the module to provide recommendations that can get ahead of fatigue by identifying changes in variables, such as sweat rate, over time that are indicative of fatigue. In one embodiment, an escalating NA+ level in the user Bauers' sweat would prompt the system to increase frequency and dose to combat the electrolyte loss continually. This module will return at step 416 to the fatigue base module if the activity is complete.

Table 2 below illustrates an exemplary set of fields in a rules database:

TABLE 2
Baseline Intervals (in minutes)

| Height | Use interval | Weight | Use Interval |
|---|---|---|---|
| <5′0″ | 12 | <100 lbs. | 12 |
| 5′0″-3″ | 12 | 101-150 lbs. | 12 |
| 5′4″-7″ | 10 | 151-200 lbs | 10 |
| 5′8″-11′ | 10 | 201-250 lbs | 10 |
| 6′0″-3″ | 8 | 250-300 lbs | 8 |
| 6′3″+ | 8 | >300 lbs | 6 |
| | High | Medium | Low |

Personalized Interval Adjusters

| | | | |
|---|---|---|---|
| Fitness level | −1 | 0 | +1 |
| Sweat level | +1 | 0 | −1 |
| Temperature | −1 | 0 | −1 |
| Humidity | 0 | 0 | −1 |
| Altitude | −1 | 0 | 0 |

Real-Time Interval Adjusters

| | | | |
|---|---|---|---|
| Heart rate | −2 | 0 | 0 |
| Salt level in sweat | −3 | −2 | 0 |

This database illustrated in Table 2 may contain the rules that govern the recommendations for use interval and dosage of an electrolyte replacement device 175 based on some amount of data about the user, the activity the user is engaged in, the context of that activity, real-time physiological data about the user, or some combination thereof. In one embodiment, users may receive a baseline use interval based on their height and weight, as a larger person will need to replace a more considerable amount of nutrients and water; when a dose remains the same, a larger user will need to use the electrolyte replacement device 175 more frequently than a smaller/HCheuser. In another embodiment, the baseline recommendation may be personalized or adjusted based on the user's number of attributes, the activity, or the context of the activity. In the present example, the user may provide a low, medium, or high fitness level. A high fitness level would indicate frequent strenuous exercise. That user would need to use the electrolyte replacement device 175 less frequently than the average individual.

Conversely, a low fitness level user would need more frequent electrolytes doses as they would be expected to sweat more than an individual of exercises routinely. A user may also identify themselves as someone with a lower or higher than average amount of sweat in response to physical exertion. The context of the activity may also serve as a metric to adjust the recommendation. For example, a user will lose more fluid in low humidity, high temperature, and high elevation. These metrics may be collected from the user through the fatigue management application 170, from the sensors on the mobile device(s) 165, or third-party providers, such as a weather network. This database may also contain adjustments that can be made based on data collected in real-time from the mobile device(s) 165 and physiological measurement device(s) 180. For example, a microfluidic patch may measure sodium and potassium levels in a user's sweat. When those levels are low, the use interval is not changed, but when those levels are high, the recommended use interval is decreased by 3 minutes. While the dosage was kept at a single uniform dose in this example, it should be obvious to one skilled in the art that dosage could be adjusted instead of, or in conjunction with, changing the use interval. Further, while the adjustments in this example are made in time increments, those adjustments could also be percentage-based, i.e., +10% instead of a one-minute increase, or scale with the measurement's amplitude. Several factors could also be used together.

Table 3 below illustrates another exemplary set of fields in a rules database:

TABLE 3

| Variable | Threshold | Notification 1 | Notification 2 |
|---|---|---|---|
| SPO2 | Less than 88% | Use Oxygen Delivery or | |
| | | rest for 5 minutes | |
| Whole-body | Greater than 6 g/min | Consume at least 12 | |
| fluid loss rate | | ounces of water | |
| Oxygen | Increase greater than 5% | For users with existing | For users without existing |
| consumption | | orders - Consider | orders - Consider ordering |
| rate change | | increasing next order | O2 |

The database illustrated in Table 3 may contain thresholds for a given variable that, when exceeded, may prompt notification to the user to hydrate, use an oxygen delivery system 120, and adjust their ordering quantity or frequency of consumables related to the use of an oxygen delivery system 120 or hydration. For example, it is well known that a person without respiratory issues should have a resting SPO2 measure of at least 95%. However, during exercise, it is understood that an SPO2 measure of between 88-92% is acceptable. The fatigue network 102 may deliver a notification to the user to use the oxygen delivery system 120 when their SPO2 level is measure at below 88% by a physiological measurement device 128 or when characteristics of the current context are correlated with an SPO2 level below 88% for a user with no physiological measurement device 128 that may measure SPO2 level. Embodiments may also include rules related to hydration in the presence of certain measures, such as whole-body fluid loss rate calculated from the data collected by a micro-fluidic sweat sensor. In one embodiment, there may be rules related to changes in the user's consumption rate of either hydration or oxygen supplies prompting a suggested change in ordering behavior.

The process begins with receiving, at step 310, a prompt from the fatigue base module 104 that data is available related to the activity of a user who has connected to the fatigue network server 110 but does not have a physiological measurement device 128 connected. The context in which the data is being received may be identified at step 402. The context may be characteristics of the user, such as age, sex, weight, etc., characteristics of the activity the user is engaged in, such as the activity type, duration, location, etc., or characteristics of the environment of the activity, such as altitude, temperature, humidity, etc. The current context characteristics are compared at step 404 to the matching characteristics in the correlation database 116. It may be determined at step 406 if the current data is correlated with a threshold value in the rules database 165. For example, the altitude (6500 feet) and activity level (running for 6 minutes) of the user may be correlated (an R-value of 0.84) with SPO2 measures (which have a rule associated with it in their value dropping below 88% rules database 165). The threshold for identifying a current context characteristic as correlated may be defined by a system administrator. For example, only correlations with an R-value greater than 0.75 may be considered correlated. In one embodiment, the threshold for correlation may be calculated by an adaptive algorithm. If no current context characteristics meet the correlation threshold, the process proceeds to step 414. The physiological data value correlated with at least one current context characteristic may be compared at step 408 to the rules database 165. It may be determined at step 410 if the current context is correlated with a data value that exceeds the threshold identified in the rules database 165. If the current context is not correlated with a physiological measure that exceeds a threshold in the rules database 165, it proceeds to step 414. Suppose the current context is correlated with a physiological measure that exceeds a threshold in the rules database 165. In that case, a notification to utilize the oxygen delivery system 120 may be delivered at step 412. The process returns to the fatigue base module 104, at step 414.

FIGS. 4A-D illustrate exemplary correlations that may be detected by a system for generating custom recommendations for fatigue management. The database may contain data that can provide relationships between characteristics of the context in which physiological data was received from a physiological measurement device 128, as calculated by the training module 120. FIGS. 4A and 4C represent examples of context characteristics, such as temperature and humidity, that is not positively correlated with a physiological measurement, such as SPO2 percentage or whole-body fluid loss rate. FIGS. 4B and 4D represent characteristics, such as altitude and temperature, that are positively correlated with a physiological measurement. In one embodiment, the relationship between context characteristics and physiological measurements may be calculated through linear regression, resulting in an R-value, which may be used as a threshold for correlation. In one embodiment, convolution may be used to examine the pattern of change over time for a given variable in a given context to determine if a similar pattern is correlated with a physiological measure. In one embodiment, the relationship between a context characteristic and a physiological measure may be filtered down to a subset of samples with a second context characteristic. For example, the correlation between SPO2 level and altitude may only have an R-value that exceeds the correlation threshold when only users between 30-35 who have run for at least 5 minutes are sampled. In one embodiment, usage and order history in the user database 114 may be correlated with the context of a user's activity. Context characteristics correlated with a change in the consumption level of oxygen may prompt the advertising module 112 to suggest a change in ordering quantity or frequency to the user.

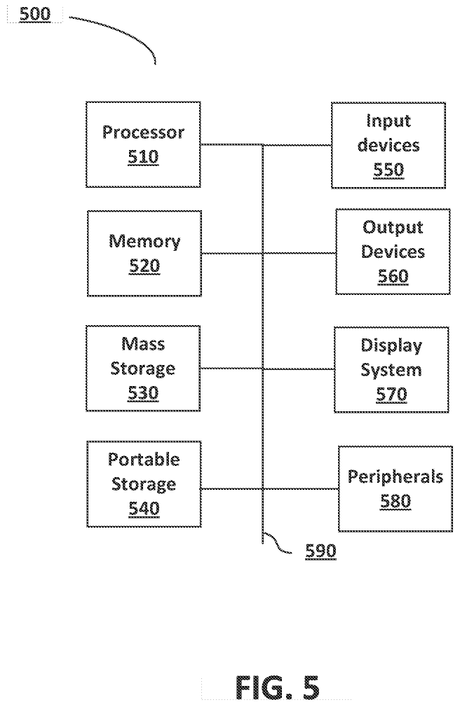

FIG. 5 is a block diagram of an exemplary computing device that may be used to implement an embodiment of the present invention. The computing system 500 of FIG. 5 includes one or more processors 510 and memory 520. Main memory 520 stores, in part, instructions and data for execution by processor 510. Main memory 520 can store the executable code when in operation. The system 500 of FIG. 5 further includes a mass storage device 530, portable storage medium drive(s) 540, output devices 550, user input devices 560, a graphics display 570, and peripheral devices 580.

The components shown in FIG. 5 are depicted as being connected via a single bus 590. However, the components may be connected through one or more data transport means. For example, processor unit 510 and main memory 520 may be connected via a local microprocessor bus, and the mass storage device 530, peripheral device(s) 580, portable storage device 540, and display system 570 may be connected via one or more input/output (I/O) buses.

Processor unit 510 may perform the operations embodied in available software. The processor unit 510 may be a digital signal controller (DSC) for processing the signals received during the methods described herein. The DSC may be a hybrid of microcontrollers and digital signal processors (DSPs). In other instances, the processor unit 510 may be a microcontroller to process the control signals received. The processor unit 510 may be manufactured by different manufacturers such as Microchip, Freescale, and Texas Instruments.

Processor unit 510 may execute instructions out of memory 520. This may include performing operations according to an algorithm that uses various criteria to generate and adjust a project assessment provided to users. Such operations and algorithms may be embodied in application software executed by the processor unit 510 at any of the local or remote computing devices of FIG. 1. The software may include a software framework or application architecture that optimizes ease of use of at least one existing software platform and may extend the capabilities of at least one existing software platform. The application architecture may approximate the actual way users organize and manage electronic files and thus may organize use activities in a natural, coherent manner while delivering use activities through a simple, consistent, and intuitive interface within each application and across applications. The architecture may also be reusable, providing plug-in capability to any number of applications without extensive reprogramming, enabling parties outside of the network environment 100 of FIG. 1 to create components that plug into the architecture. Thus, software or portals in the architecture may be extensible, and new software or portals may be created for the architecture by any party.

Main memory 520 may be used to store the software or the algorithm to perform the methods discussed herein. It can be noted that the main memory 520 may store one or more instructions and data. The data may be related to at least but not limited to various files embodying text-based documents, including agreement between lender and buyer, loan, lien documents, or loan security documents. The one or more instructions may be instructions that are executable by the processor unit 510 to perform a specific operation for performing the method for utilizing project information to adjust the credit offer of the borrower. Some of the commonly known memory implementations may include but are not limited to, fixed (hard) drives, magnetic tape, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, cloud computing platforms (e.g., Microsoft Azure and Amazon Web Services, AWS), or other types of media/machine-readable medium suitable for storing electronic instructions

Mass storage device 530, which may be implemented with a magnetic disk drive or an optical disk drive, is a non-volatile storage device for storing data and instructions for use by processor unit 510. Mass storage device 530 can store the system software for implementing embodiments of the present invention for purposes of loading that software into main memory 520.

Portable storage device 540 operates in conjunction with a portable non-volatile storage medium, such as a floppy disk, compact disk or Digital video disc, to input and output data and code to and from the computer system 500 of FIG. 5. The system software for implementing embodiments of the present invention may be stored on such a portable medium and input to the computer system 500 via the portable storage device 540.

Input devices 560 provide a portion of a user interface. Input devices 560 may include an alpha-numeric keypad, such as a keyboard, for inputting alpha-numeric and other information, or a pointing device, such as a mouse, a trackball, stylus, or cursor direction keys. Additionally, the system 500 as shown in FIG. 5 includes output devices 550. Examples of suitable output devices include speakers, printers, network interfaces, and monitors.

Input devices 560 may also be combined and operate in conjunction with output devices 550 as governed by input/output (I/O) modules. I/O modules may be used by a system administrator with specialized access. The I/O module may comprise I/O devices, display devices, or a group of devices may be augmented reality devices. The I/O devices may be controlled by an I/O controller. Furthermore, an I/O device may also allow storage or an installation medium for the computing devices 110-125. In still other embodiments, the computing devices 110-125 may allow USB connections (not shown) to receive handheld USB storage devices. In further embodiments, an I/O device may be a bridge between a system bus and an external communication bus, e.g., a USB bus, a SCSI bus, a FireWire bus, an Ethernet bus, a Gigabit Ethernet bus, a Fiber Channel bus, or a Thunderbolt bus. In some embodiments, I/O module may further include keyboards, mice, trackpads, trackballs, touchpads, touch mice, multi-touch touchpads and touch mice, pointing device, e.g., a mouse or optical pen, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors.

The I/O module may further include video displays, graphical displays, speakers, headphones, inkjet printers, laser printers, and 3D printers. Devices may include a combination of multiple input or output devices, including, e.g., Microsoft KINECT, Nintendo Wii mote for the WIT, Nintendo WII U GAMEPAD, or Apple iPhone. Some devices allow gesture recognition inputs by combining some of the inputs and outputs. Some devices allow for facial recognition, which may be utilized as an input for different purposes, including authentication and other commands. Devices allow voice recognition and inputs, including, e.g., Microsoft KINECT, SIRI for iPhone by Apple, Google Now, or Google Voice Search. Additional mobile devices have both input and output capabilities, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), or force-based sensing technologies. Some multi-touch devices may allow two or more contact points with the surface, allowing advanced functionality, including, e.g., pinch, spread, rotate, scroll, or other gestures. Further, the computing device 102 could be an optional component and would be utilized in a situation in which the paired wearable device is utilizing the computing device 110-125 as additional memory or computing power or connection to the communication network 105. It can be noted that the computing device 110-125 may be used by system administrators or by client users such as property owners, contractors, sub-contractors, borrowers, and auxiliary users associated with entities such as financial institutions and underwriters.

I/O modules may further be inclusive of network interfaces used to communicate via communication networks (e.g., communication network 105). For example, embodiments of the present invention may provide financial and site planning/progress software applications accessible to one or more computing devices 110-125, which may be associated with different users or entities to perform one or more functions. Such applications may be available at the same location or a location remote from the user. Each application may provide a graphical user interface (GUI) and a network interface for ease of interaction by the computing devices 110-125 resident in the network environment 100 and for facilitating communications therebetween. The network interface of the computing devices 110-125 may be specific to a user, set of users, or type of user, or may be the same for all users or a selected subset of users. The system software may also provide a master network interface set that allows the associated computing device 110-125 to communicate and/or interact with the network interface of one or more other applications, or that allows a computing device 110-125 to simultaneously access a variety of information otherwise available through any portion of the system. Further, the network interface may perform signal transmission and distribution functions within the computing device 110-125. The network interface may connect electronic devices or computing device 110-125 to electrical systems at a control level. Further, the network interface provides many solutions tailored for virtual network deployment and management, which efficiently optimize the distribution and management of virtual workloads and provide maximum scalability and reduced bottleneck impediments to the processes performed in network environment 100. The network interface may be easily integrated into existing hardware and architecture and configured to deploy virtual machine device queues. The network interface may be ideally suited for the consolidation of virtual network traffic without departing from the scope of the disclosure.

Display system 570 may include a liquid crystal display (LCD) or other suitable display device. Display system 570 receives textual and graphical information, and processes the information for output to the display device.

Peripherals 580 may include any type of computer support device to add additional functionality to the computer system. For example, peripheral device(s) 580 may include a modem or a router.

The components contained in the computer system 500 of FIG. 5 are those typically found in computer systems that may be suitable for use with embodiments of the present invention and are intended to represent a broad category of such computer components that are well known in the art. Thus, the computer system 500 of FIG. 5 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

While this invention has been disclosed with reference to specific embodiments, it is apparent that other embodiments and variations of this invention may be devised by others skilled in the art without departing from the true spirit and scope of the invention. It will be appreciated by those skilled in the art that changes could be made to the exemplary embodiments described above without departing from the broad inventive concept thereof. Therefore, it is to be understood that this disclosure is not limited to the particular embodiments disclosed, but it is intended to cover modifications within the spirit and scope of the subject disclosure as disclosed above.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claims.

What is claimed is:

1. A method for generating custom recommendations for managing fatigue, the method comprising:
storing one or more fatigue rules in memory, each fatigue rule correlating a set of physiological measurements to one or more actions;
monitoring sensor data received over a communication network from one or more sensors associated with a user, the sensor data including one or more physiological measurements;
identifying at least one of the fatigue rules that matches the sensor data from the one or more sensors of the users;
generating one or more custom instructions executable to initiate at an action corresponding to the identified fatigue rule; and
transmitting the generated instructions over the communication network to one or more designated user devices associated with the user.

2. The method of claim 1, wherein identifying the matching fatigue rule is further based on stored data regarding one or more characteristics associated with the user.

3. The method of claim 1, wherein generating the custom instructions is further based on stored data regarding one or more characteristics associated with the user.

4. The method of claim 1, wherein the corresponding action specifies a timing and amount selected for the user.

5. The method of claim 4, wherein the corresponding action includes a hydration recommendation based on data regarding one or more hydration resources available to the user.

6. The method of claim 4, wherein the corresponding action includes an oxygenation recommendation based on data regarding one or more oxygenation resources available to the user.

7. The method of claim 4, wherein the corresponding action includes an electrolyte recommendation based on data regarding one or more electrolyte resources available to the user.

8. The method of claim 1, further comprising continuing to monitor new sensor data in real-time, and adjusting the generated instructions based on the continued monitoring of the new sensor data.

9. A system for generating custom recommendations for managing fatigue, the system comprising:
memory that stores one or more fatigue rules, each fatigue rule correlating a set of physiological measurements to one or more actions;
a communication interface that communicates over a communication network with one or more sensors associated with a user, wherein sensor data from the sensors include one or more physiological measurements; and
a processor that executes instructions stored in memory, wherein the processor executes instructions to:
identify at least one of the fatigue rules that matches the sensor data from the one or more sensors of the users, and
generate one or more custom instructions executable to initiate at an action corresponding to the identified fatigue rule, wherein the communication interface transmits the generated instructions over the communication network to one or more designated user devices associated with the user.

10. The system of claim 9, wherein the processor identifies the matching fatigue rule further based on stored data regarding one or more characteristics associated with the user.

11. The system of claim 9, wherein the processor generates the custom instructions further based on stored data regarding one or more characteristics associated with the user.

12. The system of claim 9, wherein the corresponding action specifies a timing and amount selected for the user.

13. The system of claim 12, wherein the corresponding action includes a hydration recommendation based on data regarding one or more hydration resources available to the user.

14. The system of claim 12, wherein the corresponding action includes an oxygenation recommendation based on data regarding one or more oxygenation resources available to the user.

15. The system of claim 12, wherein the corresponding action includes an electrolyte recommendation based on data regarding one or more electrolyte resources available to the user.

16. The system of claim 9, wherein the sensors further continue to monitor new sensor data in real-time, and wherein the processor executes further instructions to adjust the generated instructions based on the continued monitoring of the new sensor data.

17. A non-transitory computer-readable storage medium, having embodied thereon a program executable by a processor to perform a method for generating custom recommendations for managing fatigue, the method comprising:
storing one or more fatigue rules in memory, each fatigue rule correlating a set of physiological measurements to one or more actions;
monitoring sensor data received over a communication network from one or more sensors associated with a user, the sensor data including one or more physiological measurements;
identifying at least one of the fatigue rules that matches the sensor data from the one or more sensors of the users;
generating one or more custom instructions executable to initiate at an action corresponding to the identified fatigue rule; and
transmitting the generated instructions over the communication network to one or more designated user devices associated with the user.
